(19) United States
(12) Patent Application Publication
(10) Pub. No.: US 2011/0053888 A1
(43) Pub. Date: Mar. 3, 2011

(54) Mixture Of Carboyhydrates And Its Use In The Preparation Of A Product Intended For Oral Or Enteral Nutrition

(75) Inventors: Angel Gil Hernandez, Badajoz (ES); Paloma San Roman Pais, Badajoz (ES); Milagros Perez Rodriguez, Badajoz (ES)

(73) Assignee: Vegenat S.A., Pueblonevo del Guadiana (ES)

(21) Appl. No.: 12/990,930

(22) PCT Filed: May 7, 2008
(86) PCT No.: PCT/ES2008/000319

§ 371 Date: Nov. 3, 2010

(51) Int. Cl. A61K 31/718; A23L 1/09; A23L 1/236; A61P 3/00; A61P 3/10; A61P 3/02

(52) U.S. Cl. 514/57; 426/661; 426/548; 426/2

(57) ABSTRACT

The present invention is related to a new mixture of carbohydrates and its usage for the preparation of a food product intended for oral or enteral nutrition, particularly for the nutrition of the diabetic patient and in particular for the nutrition of the diabetes type II patient and their associated comorbidity. The carbohydrate mixture of the present invention is formed by type IV or RS IV (modified maltodextrins) resistant starch, maltodextrin of low dextrose equivalent DE=5-8, preferably DE equal to 6, insulin and cellulose and is exempt from fructose.

The present invention refers to a new mixture of carbohydrates and its use in the preparation of a food product intended for oral or enteral nutrition, especially for the nutrition of the diabetic patient and in particular for the nutrition of the diabetes type II patient.

Whether the form of administration be oral or enteral, a large number of social groups exist which need their food requirements to be covered by this type of product. With regard to the subject under discussion, a particularly important group is that formed by mellitus diabetes patients, especially non insulin dependent mellitus diabetes or diabetes type II.

Diabetes mellitus type 2 accounts for 80-90% of diabetes cases. The frequency of diabetes mellitus is rapidly increasingly on a worldwide scale. In 1997 data regarding prevalence was around 124 million people; it is estimated that in the year 2025 this figure will have increased to 300 million. The growing proportion of diabetes type II and the fact that disorders relating to insulin resistance and its metabolic complications contribute to cardiovascular disease (Mann 2000) have increased the interest in the nutritional determinants of these metabolic complications. In this type of diabetes diverse metabolic disorders take place which are characterised by an inappropriate rise in blood glucose (hyperglycemia), which leads to chronic complications through transformation of large and small vessels and nerves. The underlying alteration in this disease is the difficulty for the insulin to act (as a loss of sensitivity of the tissues to this hormone) called “insulin resistance” and an insufficient production of insulin by the cells responsible for its production in the pancreas. As well as increasing the concentration of glucose, the defective action of the insulin frequently includes a rise in the levels of cholesterol and/or triglycerides. Most cases of diabetes mellitus type 2 occur within the context of the so-called “metabolic syndrome”, to which diabetes, high blood pressure, an increase in cholesterol, triglycerides and/or uric acid levels and overweight are associated. The metabolic syndrome notably raises cardiovascular risk and is a serious cause of death in developed countries.

The treatment of diabetes type 2 and its associated comorbidity is based upon three essential pillars: following a balanced diet plan, getting regular physical exercise and personalised pharmacological treatment.

The American Association of Diabetes and the European Association for the Study of Diabetes make a general recommendation regarding the carbohydrates present in the diet of a diabetic person: they recommend low-glycemic index foods and foods rich in soluble fibres, as well as a saccharose intake lower than 10% of the acceptable total energy in certain circumstances (FAO Food and nutrition study, 1997).

Apart from being the main substrata of energetic metabolism, carbohydrates can affect satiety, blood glucose, insulin levels, lipid metabolism and, through fermentation, exercise a major control in the functioning of the colon, including the passage through the intestines, the metabolism and balance of intestinal flora and the cellular state of the epithelium of the large intestines. They may also act as immunomodulators and influence the absorption of calcium. These properties have implications in general health, contributing, in particular, to body weight control, diabetes, cardiovascular disease, bone density, large intestine cancer, constipation and intestinal resistance to infections (Cummings, J. H., Carbohydrate: Terminology and Classification, European Journal of Clinical Nutrition, 2007).

Several controlled trials revealed that diets with a high content of soluble fibre or low-glycemic index foods are associated with an improvement in the daily glycemic profiles, and a long term improvement of glycemic control, as proven by a lower level of glycated haemoglobin (Mann, J. I., Lines to legumes: changing concepts of diabetic diets; Diabetic Medicine 1984, 1:191-198; Brand, J. C. Colagiuri, S., Crossman, S., Allen, A. Roberts, D. C., Truswell, A. S., Low-glycemic index foods improve long-term glycemic control in NIDDM; Diabetes Care 1991, 14:95-101).

In this respect, the glycemic index (GI) is a universally accepted index which is used to classify foods based on their potential to increase glycemia. The glycemic index is defined as the area of increase under the curve of glycemic response (AUC) of a portion of 50 g of carbohydrates of a trial food, expressed in percentage of response to the same amount of carbohydrate of a standard foodstuff (white bread or glucose) ingested by the same subject (Consult FAO/WHO of experts on carbohydrates in human nutrition, 1997). Monosaccharide glucose induces a high glycemic response and is the most frequently used model, being given a GI value of 100. This index has been extended to take into account the effect of the total amount of carbohydrates consumed, and, thus, the glycemic load (GL) is defined as the product of the glycemic index (GI) and the amount of carbohydrates ingested, and provides an indication of the glucose available as energy and for storage after ingestion (postprandial levels).

According to the FAO/WHO Expert Consultation on Carbohydrates in Human Nutrition (1997), foods with a low GI reduce both postprandial glycemia and insulin response. Trials with animals suggest that the incorporation of slow digestion starch into the diet delays the start of insulin resistance. Several epidemiological trials indicate that a diet with low GI is associated with a reduction in the risk of suffering from non insulin-dependent diabetes in the man and the woman. Clinical trials in normal, diabetic and hyperlipidemic subjects prove that the diets with low GI reduce average glucose concentrations levels, reduce insulin secretion and reduce the seric triglycerides in individuals with hypertriglyceridemia. Furthermore, the digestibility of carbohydrates in low GI foods is generally lower than that of foods with high GI. For this reason, foods with low GI increase the amount of carbohydrates entering into the colon and increase fermentation and the production of short chain fatty acids, which has implications for body nitrogen, lipidic metabolism and local phenomena in the colon.

There are several types of formulations on the market for oral or enteral nutrition which have been especially designed for the diabetic patient, formed by a mixture of specific nutrients and classified as “dietary products for specific nutritional uses” within the legal framework. In general, they consist of complete formulas, containing the appropriate quantity of each and every nutrient necessary for maintaining a balanced nutritional state, in supplements, which are incomplete and complement an appropriate oral diet, and, lastly, in modules which contain a specific nutrient. In the case of oral administration formulations other substances are added to improve palatability, for example flavours and aromatizers. Complete formulation products are of particular importance, where the nutritive formula contains proteins, carbohydrates, fats, fibre and micronutrients, as the T-Diet® product range by the same applicant does.

Among the said commercial products the following may be mentioned, for example: Nutrison® Diabetes (Nutricia), an enteral composition which contains a mixture of carbohydrates based on starch as the main carbohydrate and fructose (45% of the energy), as well as soluble and insoluble fibre, a high content of monounsaturated fatty acids, soya proteins and micronutrients. Diasip®, also by Nutricia, a composition for oral nutrition, contains a mixture of carbohydrates (35% of the energy) consisting of starch (6.8 g/100 ml ), fructose (1.9 g/100 ml ), saccharose (0.1 g/100 ml ) and lactose (<0.025 g/100 ml ), as well as proteins, lipids, fibre and other micronutrients; Glucerna® Select, by Abbot, a complete product for enteral or oral nutrition, containing a mixture of carbohydrates (31% of the energy) consisting of maltodextrine, modified maltodextrine, fructose, maltitol, FOS and soya polysaccharides, as well as fibre; or the Novartis Medical Nutrition range of products for the diabetic or hyperglycaemic patient, which contain carbohydrates (starch and fructose), soluble fibre, proteins and lipids.

All the previously mentioned products contain a mixture of carbohydrates the total calorific provision of which is that recommended for diabetic patients, 45-65% of the energy according to the American Diabetes Association (2000/2006) and 45-60% of the energy according to the European Association for the Study of Diabetes (1999). Fructose is included in all of them to meet with the said recommendations.

The WO2004/022074, “Nutritional compositions comprising a non-glucose carbohydrate or pectin and soluble fibre”, reveals a dietary composition which comprises carbohydrates except glucose, for example galactose, xylose, fructose or mannose, preferably galactose, in combination with soluble fibres for increasing the plasmatic levels of GLP-1 in plasma. The WO2007/004883, “A carbohydrate fraction and use thereof for a flat postprandial glucose response”, reveals a nutritional product characterised in that the carbohydrate fraction comprises: a) 5-30% in weight of one or more selected monosaccharides from between galactose, ribose and manose; b) 5-65% in weight of carbohydrates capable of providing a delayed glucose release; c) optionally fructose; d) optionally disaccharide containing glucose other than maltose and saccharose and e) optionally poliols. The ES 2211932T3, “Nutritional product for diabetics with controlled carbohydrate absorption”, is related to a nutritional composition comprising a carbohydrate component with a combination of ingredients which provide fast, moderate and show absorption of carbohydrate subsequent to consumption, resulting in a sustained carbohydrate release without excessive peaks of blood glucose, the carbohydrate component comprising between 1 and 90% of the total caloric load, wherein the said carbohydrate component comprises a quickly absorbed fraction comprising glucose, one or more disaccharides quickly absorbed which contain a unit of glucose or a mixture of the same, in which the said fraction includes saccharose; a fraction moderately absorbed which comprises one or more non glucose monosaccharides moderately absorbed, disaccharides which contain no glucose, polysaccharides which contain glucose, or mixtures of the same; and a slowly absorbed fraction which comprises one or more slowly absorbed polysaccharides which contain glucose.

Recent trials undertaken with animal models, see for example “Metabolic effects of fructose” (Lê, K-A. and Tappy, L., Current opinion in Clinical Nutrition and Metabolic Care 2006, 9: 469-475), have showed that fructose stimulates the lipogenesis and leads to hepatic and extra hepatic insulin resistance, dyslipidemia and arterial hypertension. The insulin-resistance seems to be related to ectopic fat depots. In humans, short term feeding with fructose increases the de novo lipogenesis and the blood triglyceride levels and provokes hepatic insulin resistance. The cellular mechanisms underlying the metabolic effects of fructose involve the production of reactive oxygen species, the activation of cellular stress routes and possibly an increase in uric acid synthesis. In human beings, the consumption of fructose may increase the concentrations of triglycerides in plasma and disturbs the hepatic homeostasis of glucose, although it does not appear to cause insulin-resistance in muscle nor increase blood pressure short term. John Bantle, M.D., University of Minnesota (“Effects of dietary fructose on plasma lipids in healthy subjects”, John P. Bantle, Susan K. Raatz, William Thomas and Angeliki Georgopoulos, American Journal of Clinical Nutrition, Vol. 72, N^{o} 5, 1128-1134, November 2000) carried out a trial in which two exactly the same diets were administered except that one of them contained fructose instead of starch to subjects who suffered from diabetes type I and type II. In both groups a reduction in blood glucose was confirmed. However, according to the Bantle report of November 1992 in “Diabetes Care”, total cholesterol of the subjects was raised on average by 7% and their LDL rose by almost 11%. Bantle noticed the same effects in a trial with healthy subjects. In this case, total cholesterol rose by approximately 9% and the LDL fraction by approximately 11%. Concluding that, bearing in mind the possible risk factors which these findings could involve, diets which contain fructose would not be the most appropriate for this type of patients.

The mixture of carbohydrates of this invention, apart from being new, successfully reduces the postprandial glycemic response, which is why it is useful for nutritional treatment or for the prevention of diseases associated with the metabolic syndrome in general and diabetes in particular, primordially that of diabetes type II, and its associated comorbidity, reducing the average concentrations of blood glucose, the secretion of insulin and seric triglycerides. Equally, the mixture of carbohydrates of the present invention, owing to their low glycemic index, increase the quantity of carbohydrate entering the colon and increase the fermentation and production of absorbable short chain fatty acids (SCFA), making possible the potential regulation of hepatic glyconeogenesis and insulin control, with obvious effects on the metabolism of the lipoproteins.

Therefore, the object of the present invention is to provide a mixture of carbohydrates for the nutritional treatment or for the prevention of diseases associated with the metabolic syndrome in general and diabetes in particular, primarily diabetes type II and its associated comorbidity, which will result in a reduction of the postprandial glycemic response, and which is free of fructose.

The mixture of carbohydrates of the present invention is composed by type IV or RS IV resistant starch (modified maltodextrine), maltodextrine of low dextrose equivalent DE=5-8, preferably DE equal to 6, inulin (fructooligosaccharide) and cellulose. The proportion of each one of these components present in the mixture of the invention is as follows:

Type IV resistant starch: 50.98%, soluble fibre;

maltodextrine (5-8 DE): 33.74%;

inulin: 11.74%, soluble fibre, pre-biotic carbohydrate,

Cellulose: 3.54%, insoluble fibre.

The total proportion of sugars present in the mixture of carbohydrates of the invention is approximately that of 0.91%, where the said sugars come from the maltodextrine DE 5-8 (67%) and type IV resistant starch (33%).

In one form of embodiment of the invention, the mixture of carbohydrates of the present invention additionally includes a sweetener additive, preferably potasic acesulfame and/or a stabilizer, preferably carragenate I. The proportion of these additives present in the mixture is preferably that of 0.32% for the sweetener and 0.22% for the stabilizer, percentages in weight respect to the total weight of the mixture.

The modified maltodextrine or type IV resistant starch, comprising 50.98% of the carbohydrates present in the mixture of the invention, is incorporated into the mixture of the carbohydrates of the invention in the form of the commercial product Nutriose® FM06, by the company Roquette.

### DESCRIPTION OF THE FIGURES

FIG. 1: curve relating to the glycemic index obtained with the mixture of carbohydrates of the invention in comparison with a glucose standard in accordance with one example of embodiment.

FIG. 2: curve relating to the insulin response obtained with the mixture of carbohydrates of the invention in comparison with a glucose standard in accordance with one example of embodiment.

FIG. 3: curve relating to the C-peptide obtained with the mixture of carbohydrates of the invention in comparison with a glucose standard in accordance with one example of embodiment.

FIG. 4: curve relating to the triglyceride level obtained with the mixture of carbohydrates of the invention in comparison with a glucose standard in accordance with one example of embodiment.

FIG. 5: curve relating to the glycemic index obtained with a food product containing the mixture of carbohydrates of the invention corresponding to a comparative trial with two commercial products in a clinical trial with diabetic volunteers.

FIG. 6: curve relating to the triglyceride level obtained with a food product containing the mixture of carbohydrates of the invention corresponding to a comparative trial with two commercial products in a clinical trial with diabetic volunteers.

FIG. 7: curve relating to the insulin response obtained with a food product containing the mixture of carbohydrates of the invention corresponding to a comparative trial with two commercial products in a clinical trial with diabetic volunteers.

FIG. 8: curve relating to the C-peptide obtained with a food product containing the mixture of carbohydrates of the invention corresponding to a comparative trial with two commercial products in a clinical trial with diabetic volunteers.

FIG. 9: curve relating to the ghrelin obtained with a food product containing the mixture of carbohydrates of the invention corresponding to a comparative trial with two commercial products in a clinical trial with healthy and diabetic volunteers.

FIG. 10: curve relating to the leptin obtained with a food product containing the mixture of carbohydrates of the invention corresponding to a comparative trial with two commercial products in a clinical trial with healthy and diabetic volunteers

FIG. 11: curves relating to the intestinal peptides, GIP-total, GLP-1 active, PP and PYY peptide obtained with a food product containing the mixture of carbohydrates of the invention corresponding to a comparative trial with two commercial products in a clinical trial with healthy and diabetic volunteers.

In an embodiment example, the mixture of carbohydrates of the invention is made up of the ingredients and quantities of the same shown in the following Table 1:

TABLE 1

| | Quantity | Proteins | HC+ fibre | Sugars | Lipids |
|---|---|---|---|---|---|
| Ingredients | (g) | (g) | (g) | (g) | (g) |
| Nutriose FM06 | 4.91 | 0.01473 | 4.17 | 0.02455 | 0.00491 |
| Maltodextrine | 2.90 | — | 2.76 | 0.05 | — |
| 5-8 DE | | | | | |
| Insulin | 0.99 | — | 0.96 | 0.00 | — |
| Cellulose | 0.30 | — | 0.29 | — | — |
| Ace-K | 0.03 | — | — | — | — |
| Carragenate I | 0.02 | — | — | — | — |
| Total | 9.15 | 0.01473 | 8.18 | 0.07455 | 0.00491 |

In this embodiment example, the amounts indicated correspond with the percentages of ingredients present in the mixture indicated in Table 2:

TABLE 2

| Ingredients | % |
|---|---|
| Nutriose FM06 | 53.66 |
| Maltodextrine 5-8 DE | 31.70 |
| Insulin | 10.81 |
| Cellulose | 3.28 |
| Ace-K | 0.33 |
| Carragenate I | 0.22 |
| Total | 100 |

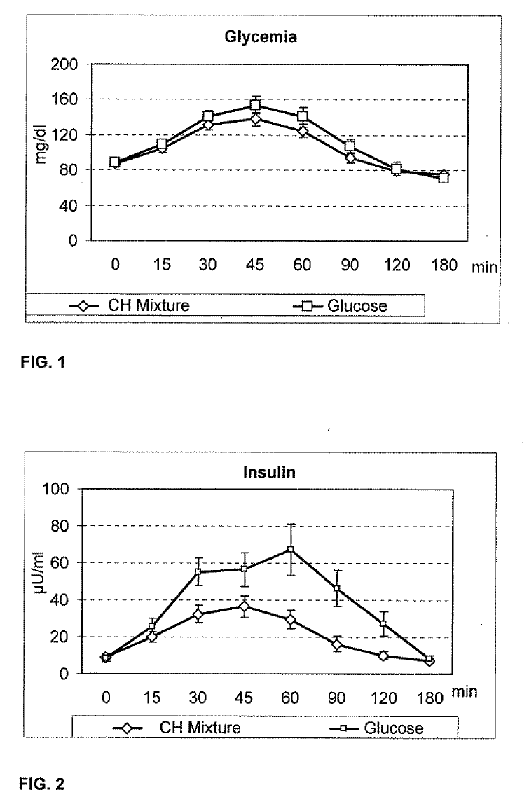

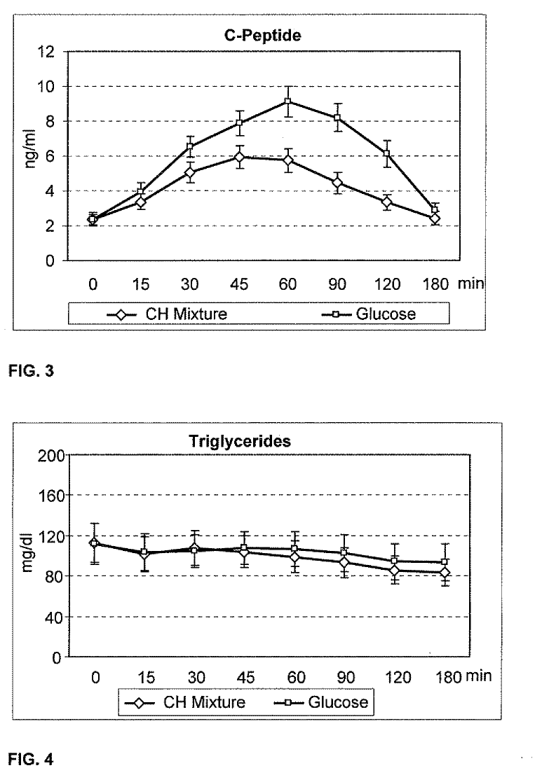

A trial was made with healthy volunteers to which the mixture of carbohydrates as of the present invention was administered orally. The findings obtained are revealed below (FIGS. 1 to 4):

| | | CH Mixture | | | |
|---|---|---|---|---|---|
| | | as of | | 50 g of | |
| | Time | the invention | | Glucose | |
| | (min) | Mean | SEM | Mean | SEM |
| Glucose | 0 | 87.5 | 2.4 | 88.1 | 3.4 |
| | 15 | 104.5 | 4.2 | 109.8 | 5.8 |
| | 30 | 131.5 | 5.5 | 140.4 | 7.6 |
| | 45 | 138.0 | 7.5 | 154.0 | 9.7 |
| | 60 | 125.0 | 7.9 | 140.6 | 11.0 |
| | 90 | 94.1 | 6.3 | 107.1 | 8.2 |
| | 120 | 78.7 | 2.0 | 81.9 | 7.6 |
| | 180 | 75.6 | 2.0 | 70.4 | 1.9 |
| Insulin | 0 | 9.2 | 1.4 | 8.2 | 1.6 |
| | 15 | 20.0 | 2.8 | 25.4 | 4.5 |
| | 30 | 32.4 | 4.7 | 55.3 | 7.7 |
| | 45 | 36.4 | 6.0 | 56.4 | 9.3 |
| | 60 | 29.4 | 5.2 | 67.3 | 13.7 |
| | 90 | 16.3 | 4.0 | 46.4 | 9.6 |
| | 120 | 10.3 | 2.0 | 27.1 | 6.7 |
| | 180 | 7.4 | 1.3 | 8.4 | 1.7 |
| C- | 0 | 2.4 | 0.4 | 2.4 | 0.3 |
| Peptide | 15 | 3.4 | 0.4 | 3.9 | 0.5 |
| | 30 | 5.1 | 0.6 | 6.5 | 0.6 |
| | 45 | 5.9 | 0.7 | 7.9 | 0.7 |
| | 60 | 5.7 | 0.7 | 9.1 | 0.9 |
| | 90 | 4.5 | 0.6 | 8.2 | 0.8 |
| | 120 | 3.3 | 0.5 | 6.1 | 0.8 |
| | 180 | 2.4 | 0.4 | 2.9 | 0.4 |
| TAG | 0 | 112.5 | 19.6 | 111.6 | 20.6 |
| | 15 | 101.2 | 17.1 | 103.3 | 18.4 |
| | 30 | 107.4 | 17.2 | 104.8 | 16.4 |
| | 45 | 104.0 | 16.0 | 107.5 | 16.0 |
| | 60 | 98.8 | 15.9 | 106.8 | 17.4 |
| | 90 | 92.9 | 14.8 | 102.8 | 18.3 |
| | 120 | 85.5 | 13.6 | 94.0 | 18.1 |
| | 180 | 83.3 | 12.9 | 93.1 | 18.3 |

TAG = Triacilglycerides

The findings for the area under the glycemic response curve obtained in the subjects of the trial are contained in the following Table 3:

TABLE 3

| | CH Mixture as | | |
|---|---|---|---|
| | of the invention | 50 g of Glucose | |
| | AUC | AUC | |
| | (Mean ± SEM) | (Mean ± SEM) | Value p¹⁾ |
| Glucose 120 min | 53 ± 14 | 62 ± 17 | 0.05 |
| (mmol/l) | | | |
| Insulin 120 min | 1275 ± 323 | 3917 ± 603 | 0.001 |
| C-Peptide 120 min | 263 ± 23 | 605 ± 44 | 0.001 |
| Triglycerides 120 min | −1056 ± 287 | −1367 ± 510 | 0.813 |

¹⁾comparison of means: test T of Student. Significance level 95%

The following conclusions may be drawn, based on these findings:
The glycemic index (GI) of the carbohydrate mixture as of the invention, taking 50 g of Glucose as standard, is the following:

$$GI ( \% ) = \frac{AUC CH mixture of the invention}{AUC standard} \times 100 = 85 , 48 \%$$

The glycemic load (GL) of the carbohydrate mixture as of the invention, taking 50g of glucose as the standard is 7.7 g/100 ml.
The calculation relating to the insulin response gives the following result:

AUC Insulin=32.50%
The area below the curve (AUC) relating to the C-Peptide gives the following result

AUC C-Peptide=43.50%

As may be drawn from previous data, the carbohydrate mixture of the present invention successfully reduces the postprandial glycemic response, reducing the average glucose in blood concentrations and the secretion of insulin. Equally, owing to the fact it does not contain fructose, higher clearing of circulating lipids may be made. The carbohydrate mixture, therefore, as of the invention is useful for the nutritional treatment or for the prevention of diseases associated with the metabolic syndrome in general and diabetes in particular, primarily in type II diabetes. Equally, the carbohydrate mixture of the present invention increases the quantity of carbohydrates entering the colon and increases the fermentation and production of absorbable short chain fatty acids (SCFA), leading to the potential regulating of hepatic glyconeogenesis and insulin control, with obvious effects on the metabolism of the lipoproteins.

A comparative trial was also carried out with diabetic volunteers to whom the carbohydrate mixture of the present invention was orally administered.

The said study was carried out on 10 subjects aged between 18 and 60 years of age, 4 men and 6 women, clinically stable, diagnosed as having type II diabetes and who were treated with insulin or with oral antidiabetics (OAD) during the trial. The subjects of the trial were orally administered with 300 ml of a food product containing the carbohydrate mixture of the present invention, in the morning before ingesting anything, or with the same amount of two commercial products adapted for the nutrition of the diabetic patient (Glucerna®, by Abbot Laboratories, and Novasource®, by Nestlé). In order to determine the expression of the metabolism of the carbohydrates in the subjects the following analytic determinations were carried out for three weeks in the instances T=0 (base), 15, 30, 45, 60, 90, 120 and 180 minutes following administration: Glucose (Glu), triglycerides (TG), plasmatic Insulin and C-Peptide.

In Table 4 below the differential characteristics of the 3 trial products are detailed: the product which contains the carbohydrate mixture of the present invention and the two before-mentioned commercial products.

TABLE 4

| | Product | | |
|---|---|---|---|
| | containing the | | |
| | CH mixture of the | | |
| | present invention | Glucerna ® | Novasource ® |
| VCT (kcal/ml) | 1.0 | 0.9 | 1.0 |
| I P/CH/G (%) | 23:32:45 | 21:45:34 | 28:47:25 |
| Profile | | | |
| Proteins (g %) | 5.7 | 4.65 | 7.0 |
| Carbohydrates | 8.0 | 11.1 | 11.8 |
| (g %) | | | |
| Of which | 54 mod. maltodext. | 7 mod. maltodext. | 80 starch |
| (%) | 32 maltodextrine | 43 maltodextrine | 20 fructose |
| | 11 insulin | 23 fructose | |
| | 3 cellulose | 20 maltitol | |
| Fats (g %) | 5.1 | 3.4 | 2.8 |
| Fatty acids (%) | 19 AGS | 9 AGS | 18 AGS |
| | 55 AGMI | 76 AGMI | 54 AGMI |
| (g %) | 26 AGPI | 15 AGPI | 28 AGPI |
| MCT | 0.5 | | 0.5 |
| Linolenic Acid | 1.01 | | |
| Linolenic acid | 0.12 | | |
| EPA-DHA | 0.02 | | |
| | (EPA/DHA 2:1) | | |
| Dietary fibre | 1.8 | 0.76 | 2.0 |
| (g %) | | | |
| 100% RDA | 1.500 | 1.024 | 1.500 |
| vitamins and | | | |
| minerals (kcal) | | | |

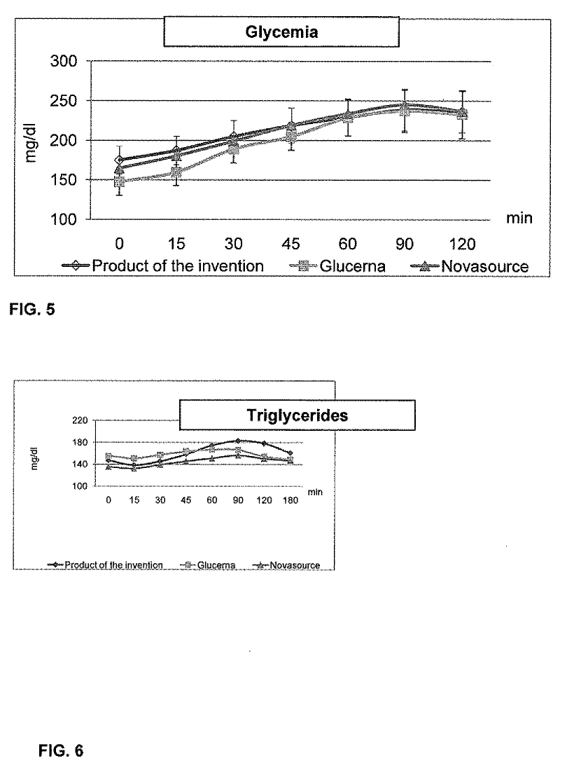

The findings obtained for the increase of the area under the curve (IABC) in t=120 min for the glucose and in t=180 min for the Insulin, the C-Peptide and the triglycerides are shown below (see FIGS. 5, 6, 7 and 8).

| | Product | | |
|---|---|---|---|
| | containing the | | |
| | CH mixture of the | | |
| IABC (X ± EEM) | present invention | Glucerna ® | Novasource ® |
| Glucose | 297 ± 62.0 | 412 ± 70.9 | 372 ± 69.2 |
| (mg/min/ml) | | | |
| Insulin (μU/min/ml) | 1758 ± 640 | 2578 ± 717 | 2578 ± 717 |
| C-Peptide | 232 ± 55.5 | 277 ± 76.2 | 269 ± 62.2 |
| (ng/min/ml) | | | |
| Triglycerides | 3832 ± 876 | 1428 ± 472 | 3131 ± 788 |
| (mg/min/ml) | | | |

*p < 0.05: statistically significant difference (Willcoxon Test)

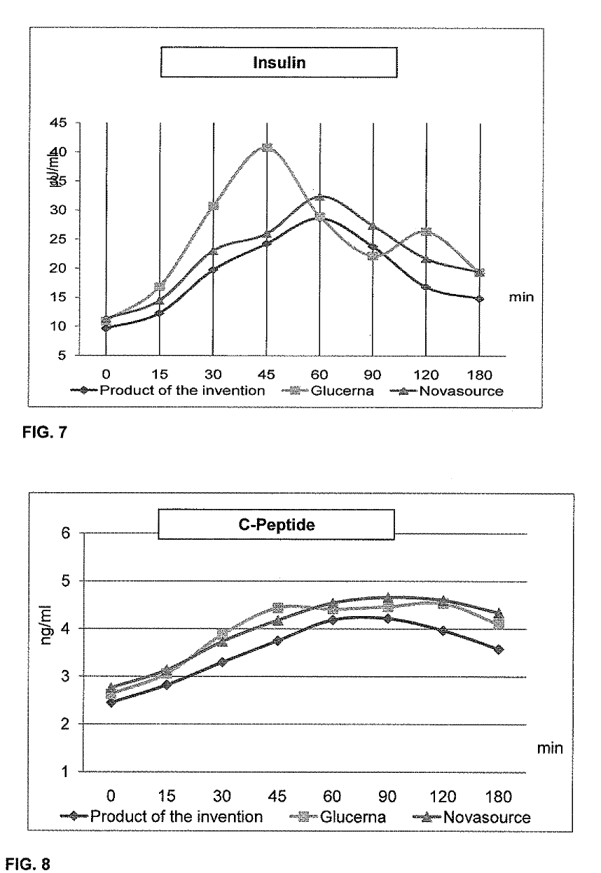

As may be observed in the previous Table and in FIG. 5, the product which contains the carbohydrate mixture of the present invention successfully reduces the postprandial glycemic response, as do the two products included for comparative reasons, to a different degree, reducing the average blood glucose concentrations to similar levels. With regards to the insulin response, as is showed in FIG. 7 and in the previous table, the product containing the carbohydrate mixture of the invention leads to a lower secretion of insulin than the commercial products, the insulin response curve being gentler and more constant over time. Thus, the carbohydrate mixture as of the invention and the food product containing it reduce the glucose curve without the need to administer fructose, in comparison to the commercial products Glucerna® and Novasource®, maintaining comparable levels of insulinemia and other peptides involved in the control of insulin synthesis. To conclude, the mixture of carbohydrates as of the invention and the food product containing it demonstrate how ideal they are in the nutritional treatment or prevention of diseases associated with the metabolic syndrome in general and with diabetes in particular, primarily type II diabetes.

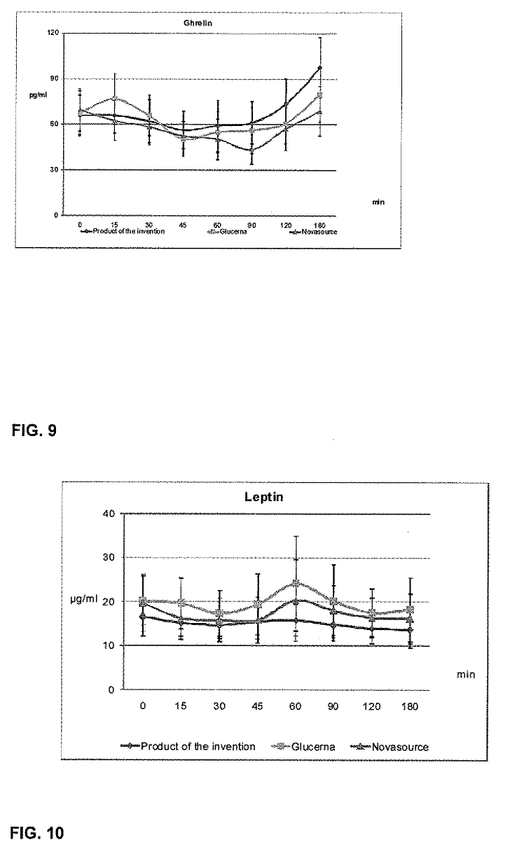

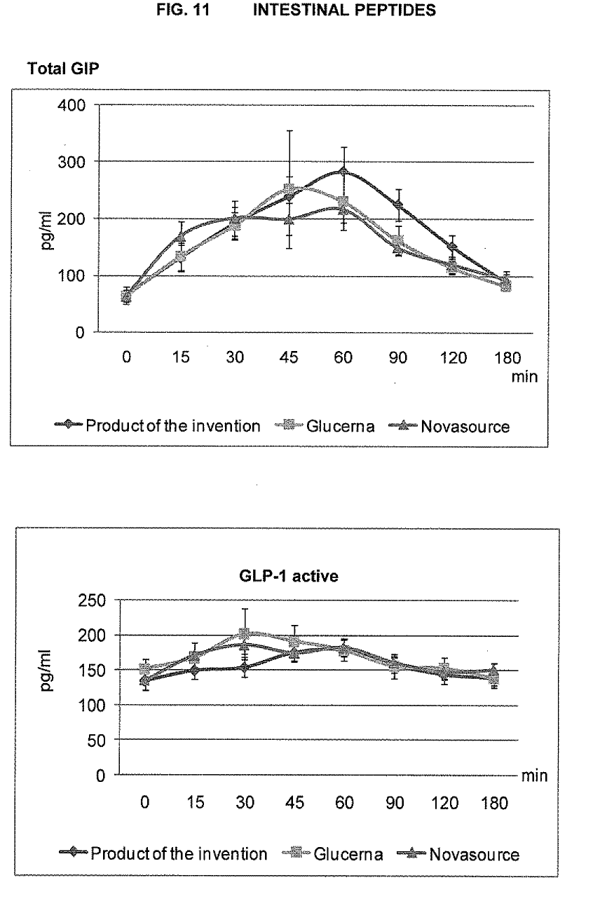

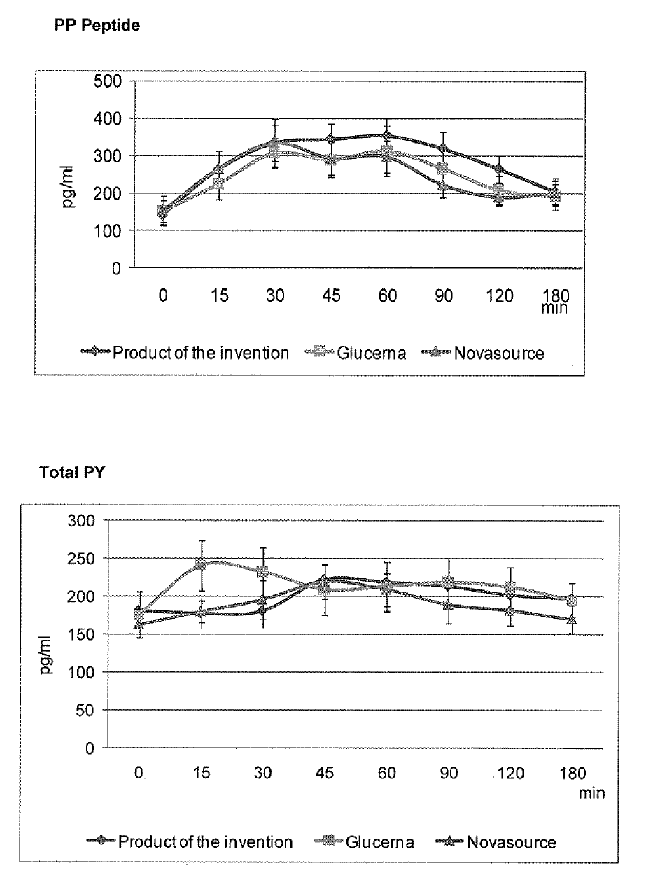

A comparative trial was also carried out in order to determine the levels of the two hormones involved in regulating appetite and satiety: ghrelin and leptin. Ghrelin, synthesized in the stomach, stimulates hunger and encourages ingestion of food, whilst leptin, synthesized in the adipose tissues, increases satiety and reduces food consumption. The responses of ghrelin and leptin were researched in this trial, as were the intestinal peptides regulators of satiety, GLP-1, GIP, PYY and PP, in food ingestion of patients diagnosed with type II diabetes with reference to the product containing the carbohydrate mixture of the invention and compared with the two commercial products previously used, Glucerna® y Novasource®. In this case, the trial parameters were the same as in the previously described trial with individuals diagnosed with type II diabetes and treated with OADs. The limitation of the sample size of the groups prevented any definitive conclusions being made, but indicate that with the administration of the food product containing the carbohydrate mixture of this invention suitable glycemic control could be made, as could that of the gastrointestinal peptides involved in regulating insulin secretion and in appetite-satiety control, as may be observed in FIGS. 9, 10 and 11.

### Haber Satiety Test

In order to confirm the previously showed findings, a Haber satiety test was undertaken with both the healthy and diabetic subjects participating in the trials that have already been described. Either a placebo product was administered to the subjects or one of the commercial products Glucerna® or Novasource® and their subjective satiety-hunger consideration was compared with that experienced with the product containing the mixture pertaining to the invention. To do this they were requested to give a score from −10 (extremely hungry)/−8/−6/−4/0 (no hunger)/+2/+4/+6/+8/ to +10 (completely satisfied) indicating their sensation of satiety-hunger. The findings were obtained as the sum of the scores of each subject in 8 controls and a Willcoxon (p>0.05) test was completed. The findings subjectively corroborated those previously indicated, which reaffirms that with the administration of the food product containing the carbohydrate mixture of the present invention appropriate control may be made of glycaemia and the gastrointestinal peptides involved in regulating insulin secretion and in the control of the appetite-satiety sensation.

1. Mixture of carbohydrates for its use in a food product intended for enteral or oral nutrition characterised in that it contains type IV resistant starch, maltodextrin of low dextrose equivalent DE=5-8, insulin and cellulose.

2. Mixture of carbohydrates according to claim 1, characterised in that it contains 50.98% in weight of type IV resistant starch, 3374% in weight of maltodextrin 5-8 DE, 11.74% in weight of insulin and 3.54% in weight of cellulose, percentages respect to the total weight of the mixture.

3. Mixture of carbohydrates according to claim 1, characterised in that it is exempt from fructose.

4. Mixture of carbohydrates according to claim 1, characterised in that it also contains at least one additive selected from among sweeteners and stabilizers.

5. Mixture of carbohydrates according to claim 4, characterised in that the proportion of sweetener is that of 0.32% in weight with respect to the total weight of the mixture.

6. Mixture of carbohydrates according to claim 4, characterised in that the proportion of stabiliser is 0.22% in weight with respect to the total weight of the mixture.

7. Mixture of carbohydrates according to claim 1, characterised in that the maltodextrin of low glucose equivalent has a DE equal to 6.

8. Mixture of carbohydrates according to claim 2, characterised in that it contains sugars of 0.91% with respect to the total weight of the mixture.

9. Mixture of carbohydrates according to claim 1 characterised in that it has a glycemic index of 85.48% mmol/l.

10. A method for enteral or oral nutrition characterised by consuming a food product that includes a mixture of carbohydrates according to claim 1.

11. A method of nutritional treatment or the prevention of diseases associated with the metabolic syndrome in general and diabetes in particular, primarily type II diabetes and its associated comorbidity, characterised by consuming a food product including a mixture of carbohydrates according to claim 1.
